(19) United States
(12) Patent Application Publication
(10) Pub. No.: US 2026/0097057 A1
(43) Pub. Date: Apr. 9, 2026

(54) COMBINATION OF ZIBOTENTAN AND DAPAGLIFLOZIN FOR THE TREATMENT OF CHRONIC KIDNEY DISEASE AND LIVER FIBROSIS

(71) Applicant: AstraZeneca AB, Södertälje (SE)

(72) Inventor: Philip Ambery, Södertälje (SE)

(21) Appl. No.: 19/352,788

(22) Filed: Oct. 8, 2025

Related U.S. Application Data
(60) Provisional application No. 63/705,276, filed on Oct. 9, 2024.
(60) Provisional application No. 63/738,901, filed on Dec. 26, 2024.
(60) Provisional application No. 63/828,388, filed on Jun. 23, 2025.

(51) Int. Cl. A61K 31/7034; A61K 31/497; A61P 1/16

(52) U.S. Cl. CPC A61K 31/7034; A61K 31/497; A61P 1/16

(57) ABSTRACT

Methods for treating chronic kidney disease and liver fibrosis in patients using a fixed-dose combination of zibotentan and dapagliflozin are disclosed.

### CROSS-REFERENCE TO RELATED APPLICATIONS

This application is filed under 35 U.S.C. § 111 (a) and claims priority under 35 U.S.C. § 119 (e) to U.S. Provisional Application No. 63/705,276, filed Oct. 9, 2024, U.S. Provisional Application No. 63/738,901, filed Dec. 26, 2024, and U.S. Provisional Application No. 63/828,388, filed Jun. 23, 2025. The contents of these applications are each incorporated herein by reference in their entirety.

### TECHNICAL FIELD

The present disclosure relates to a fixed-dose combination of the endothelin receptor antagonist (ERA), zibotentan, and the sodium-dependent glucose cotransporter 2 (SGLT2) inhibitor, dapagliflozin, for use in the treatment of chronic kidney disease and liver fibrosis.

### INTRODUCTION

Chronic kidney disease (CKD) is an endothelin-related disease associated with the gradual loss of kidney function. In some instances, CKD may be associated with hypertension or diabetes (Diabetic Kidney Disease, DKD). CKD may be diagnosed by measuring the estimated glomerular filtration rate (eGFR) in the blood, and by measuring albumin and/or protein levels in urine. The severity of CKD is determined by a patient's eGFR levels that correspond with a given stage of disease, ranging from stage 1 (eGFR≥90 mL/min/1.73 m²; normal) to stage 5 (eGFR<15 mL/min/1.73 m²; kidney failure). When diagnosing CKD, albuminuria may also be categorized as an indicator of disease progression. A urine albumin to creatine ratio (UACR) of <30 mg/g is categorized as normal, a UACR between 30-300 mg/g is categorized as moderately increased, and a UACR that is >300 mg/g is categorized as severely increased. There is no cure for, nor way to reverse CKD, and current treatments focus on slowing the progression of kidney damage and controlling symptoms.

Liver fibrosis results from the accumulation of scar tissue, collagen, and/or other extracellular matrix proteins that occur in many types of chronic liver disease, including steatosis. Metabolic dysfunction-associated steatotic liver disease (MASLD) is a disease caused by excessive buildup of fat in the liver that develops without the use of alcohol. MASLD is estimated to affect up to 25% of people worldwide, and progression of MASLD results in metabolic dysfunction-associated steatohepatitis (MASH), where inflammation causes cell damage in the liver, and may result in fibrosis. Current treatments for MASLD and MASH include weight loss (e.g., through diet and exercise), and although the FDA only recently approved resmetirom for the treatment of MASH, there is no FDA-approved treatment to reverse MASLD (Keam S J, Resmetirom: first approval, Drugs, 2024, 84 (6): 729-735).

Endothelin-1 (ET-1) is a highly potent systemic vasoconstrictor and driver of renal disease progression that is modulated by endothelin A and B receptors (ET_{A} and ET_{B}). In CKD, ET-1 levels increase with UACR and severity of renal functional impairment (Grenda et al., Nephrol Dial Transplant. 2007; 22 (12): 3487-3494; Kohan Am J Kidney Dis. 1997; 29 (1): 2-26). The pathological effects of ET-1 accumulation, including proteinuria, vasoconstriction, and inflammation are thought to be predominantly driven by the ET_{A} receptor (Goddard et al., Circulation. 2004; 109 (9): 1186-1193). ET_{A} receptor antagonists have demonstrated kidney protective effects but have side-effects including edema (swelling). In Diabetic Kidney Disease (DKD), short-term treatment with an ET_{A} receptor antagonist has demonstrated a 30% reduction in urinary albumin to creatinine ratio (UACR) (Heerspink et al., Diabetes Obes Metab. 2018; 20 (8): 1829-1835; Heerspink et al., Lancet 2019; 393 (10184): 1937-1947). However, clinical development of ET_{A} receptor antagonists have been limited due to issues of fluid retention and hospitalization for heart failure (Heerspink et al., Lancet 2019; 393 (10184): 1937-1947).

Zibotentan is an ET_{A} receptor antagonist developed for treatment of prostate cancer but was abandoned in 2011 due to insufficient efficacy in Phase 3 and a 17% increase in incidence of peripheral oedema compared to placebo. Zibotentan, also referred to as ZD4054, was described in WO1996040681 along with details on its chemical synthesis, and those teachings are incorporated herein by reference. The specific inhibition of the endothelin A receptor with zibotentan has been reported by Morris et al., British Journal of Cancer (2005), 92, 2148-2152. Zibotentan, N-(3-methoxy-5-methylpyrazin-2-yl)-2-[4-(1,3,4-oxadiazol-2-yl)phenyl]pyridine-3-sulfonamide, has the chemical structure of formula I:

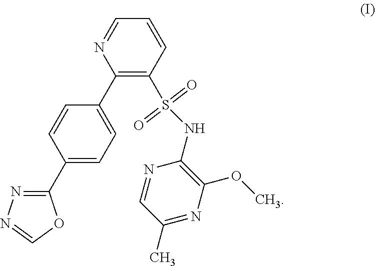 [SMILES: COc1nc(C)cnc1NS(=O)(=O)c1cccnc1-c1ccc(-c2nnco2)cc1]

Sodium-dependent glucose transporter 2 (SGLT-2) inhibitors result in osmotic diuresis and block glucose reabsorption in the kidney, increase glucose excretion, and lower blood glucose concentration. In addition to this well characterized mode of action, SGLT-2 inhibitors reduce blood pressure, decrease vascular stiffness, improve endothelial function, and have anti-inflammatory and anti-fibrotic properties resembling those of endothelin receptor antagonists (H. J. Heerspink et al., Circulation (2016), 134 (10): 752-772). Like ET_{A} receptor antagonists, SGLT2 inhibitors have demonstrated efficacy in reducing the progression of DKD (Stephens et al., Diabetes Obes Metab. 2020; 22 Suppl 1:32-45). A side effect associated with the pharmacological effects of SGLT-2 inhibitors is volume depletion/intravascular volume contraction, potentially leading to dehydration, hypovolemia, orthostatic hypotension, or hypotension. Thus, SGLT-2 inhibitors generally induce an increase in hematocrit (Hot) a marker of hemoconcentration and increased blood viscosity, a putative cause of vascular injury in a context of peripheral vascular disease.

Dapagliflozin is a potent, highly selective, and orally active inhibitor of human renal SGLT2 that has been approved to improve glycemic control in adults with type 2 diabetes mellitus (as an adjunct to diet and exercise). Dapagliflozin has been disclosed in WO2003099836 along with details on its chemical synthesis, and those teachings are incorporated herein by reference.

Dapagliflozin, whose IUPAC name is (1S)-1,5-anhydro-1-{4-chloro-3-[(4-ethoxyphenyl)methyl]phenyl}-D-glucitol, has the chemical structure of formula II:

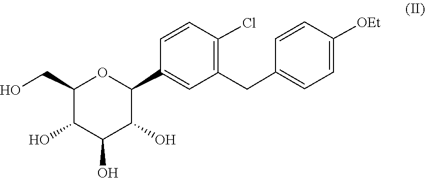 [SMILES: CCOc1ccc(Cc2cc([C@@H]3O[C@H](CO)[C@@H](O)[C@H](O)[C@H]3O)ccc2Cl)cc1]

While approved therapies exist for patients having CKD, the impact of these therapies on conditions that may be associated with CKD, such as liver fibrosis, remain unknown. Further, increased doses of an ET_{A} receptor antagonist that would be required to treat more severe stages of CKD are not well tolerated due to side-effects including edema and risk of congestive heart failure. SGLT2 inhibitors, while effective at treating CKD in many circumstances, leave residual risk in many patients as evidenced by continued proteinuria. For example, a post hoc analysis of the CREDENCE trial showed that while canagliflozin, a SGLT2 inhibitor for treatment of renal events in patients with type 2 diabetes and CKD, was able to reduce UACR levels in the short term, it failed to reduce UACR levels below 300 mg/g in a substantial number of patients and these patients developed kidney events and major adverse cardiovascular events due to residual proteinuria (Oshima et al., J Am Soc Nephrol. (2020); 31 (12): 2925-2936). Additionally, none of these studies have further investigated a link between CKD and liver fibrosis. Specifically, there remains a need for improved compounds, compositions, and methods for treating patients with CKD and liver fibrosis. The present disclosure addresses those unmet needs.

### BRIEF SUMMARY

The present disclosure provides methods of treating liver fibrosis in a human patient who has chronic kidney disease (CKD), the method comprising administering to the patient a fixed-dose combination of zibotentan and dapagliflozin in an amount effective to treat the patient's liver fibrosis. In some embodiments, the patient is suspected of having liver fibrosis. The present disclosure provides methods of reducing a urine albumin-creatinine ratio (UACR) and for treating liver fibrosis in a human patient who has chronic kidney disease (CKD), the method comprising administering to the patient a fixed-dose combination of zibotentan and dapagliflozin in an amount effective to treat the patient's liver fibrosis and to reduce the patient's UACR.

The present disclosure also provides a fixed-dose combination of zibotentan and dapagliflozin for use in treating liver fibrosis in a human patient having CKD and liver fibrosis. The present disclosure also provides a fixed-dose combination of zibotentan and dapagliflozin for use in reducing a urine albumin-creatinine ratio (UACR) and for treating liver fibrosis in a human patient having CKD and liver fibrosis.

The present disclosure also provides the use of zibotentan and dapagliflozin in the manufacture of a medicament for treating liver fibrosis in a human patient having CKD and liver fibrosis, wherein a fixed-dose combination of zibotentan and dapagliflozin is administered. The present disclosure also provides the use of zibotentan and dapagliflozin in the manufacture of a medicament for reducing a urine albumin-creatinine ratio (UACR) and for treating liver fibrosis in a human patient having CKD and liver fibrosis, wherein a fixed-dose combination of zibotentan and dapagliflozin is administered.

The present disclosure also provides methods of slowing liver fibrosis in a human patient who has chronic kidney disease (CKD), the method comprising administering to the patient a fixed-dose combination of zibotentan and dapagliflozin in an amount effective to slow the patient's liver fibrosis relative to a dosing regimen in which the patient receives dapagliflozin alone. The present disclosure also provides methods of reducing a urine albumin-creatinine ratio (UACR) and slowing liver fibrosis in a human patient who has chronic kidney disease (CKD), the method comprising administering to the patient a fixed-dose combination of zibotentan and dapagliflozin in an amount effective to slow the patient's liver fibrosis and to reduce the patient's UACR relative to a dosing regimen in which the patient receives dapagliflozin alone.

The present disclosure also provides a fixed-dose combination of zibotentan and dapagliflozin for use in slowing liver fibrosis in a human patient having CKD and liver fibrosis. The present disclosure also provides a fixed-dose combination of zibotentan and dapagliflozin for use in reducing a urine albumin-creatinine ratio (UACR) and slowing liver fibrosis in a human patient having CKD and liver fibrosis.

The present disclosure also provides the use of zibotentan and dapagliflozin in the manufacture of a medicament for slowing liver fibrosis in a human patient having CKD and liver fibrosis, wherein a fixed-dose combination of zibotentan and dapagliflozin is administered. The present disclosure also provides the use of zibotentan and dapagliflozin in the manufacture of a medicament for reducing a urine albumin-creatinine ratio (UACR) and slowing liver fibrosis in a human patient having CKD and liver fibrosis, wherein a fixed-dose combination of zibotentan and dapagliflozin is administered.

The present disclosure also provides methods of slowing nonalcoholic fatty liver disease (NAFLD) or cirrhosis in a human patient who has chronic kidney disease (CKD) and liver fibrosis, the method comprising administering to the patient a fixed-dose combination of zibotentan and dapagliflozin in an amount effective to slow the patient's NAFLD or cirrhosis relative to a dosing regimen in which the patient receives dapagliflozin alone. In at least one embodiment, slowing NAFLD or cirrhosis can be measured by a reduction in fibrosis score (NFS).

The present disclosure also provides a fixed-dose combination of zibotentan and dapagliflozin for use in slowing nonalcoholic fatty liver disease (NAFLD) or cirrhosis in a human patient having CKD and liver fibrosis. In at least one embodiment, slowing NAFLD or cirrhosis can be measured by a reduction in fibrosis score (NFS).

The present disclosure also provides the use of zibotentan and dapagliflozin in the manufacture of a medicament for slowing nonalcoholic fatty liver disease (NAFLD) or cirrhosis in a human patient having CKD and liver fibrosis, wherein a fixed-dose combination of zibotentan and dapagliflozin is administered. In at least one embodiment, slowing NAFLD or cirrhosis can be measured by a reduction in fibrosis score (NFS).

The present disclosure also provides methods of reducing a nonalcoholic fatty liver disease (NAFLD) fibrosis score (NFS) in a human patient who has chronic kidney disease (CKD) and liver fibrosis, the method comprising administering to the patient a fixed-dose combination of zibotentan and dapagliflozin in an amount effective to reduce the patient's NFS score relative to a dosing regimen in which the patient receives dapagliflozin alone. The present disclosure also provides methods of reducing a urine albumin-creatinine ratio (UACR) and a nonalcoholic fatty liver disease (NAFLD) fibrosis score (NFS) in a human patient who has chronic kidney disease (CKD) and liver fibrosis, the method comprising administering to the patient a fixed-dose combination of zibotentan and dapagliflozin in an amount effective to reduce the patient's NFS score and to reduce the patient's UACR relative to a dosing regimen in which the patient receives dapagliflozin alone.

The present disclosure also provides a fixed-dose combination of zibotentan and dapagliflozin for use in reducing a nonalcoholic fatty liver disease (NAFLD) fibrosis score (NFS) in a human patient having CKD and liver fibrosis. The present disclosure also provides a fixed-dose combination of zibotentan and dapagliflozin for use in reducing a urine albumin-creatinine ratio (UACR) and reducing a nonalcoholic fatty liver disease (NAFLD) fibrosis score (NFS) in a human patient having CKD and liver fibrosis.

The present disclosure also provides the use of zibotentan and dapagliflozin in the manufacture of a medicament for reducing a nonalcoholic fatty liver disease (NAFLD) fibrosis score (NFS) in a human patient having CKD and liver fibrosis, wherein a fixed-dose combination of zibotentan and dapagliflozin is administered. The present disclosure also provides the use of zibotentan and dapagliflozin in the manufacture of a medicament for reducing a urine albumin-creatinine ratio (UACR) and reducing a nonalcoholic fatty liver disease (NAFLD) fibrosis score (NFS) in a human patient having CKD and liver fibrosis, wherein a fixed-dose combination of zibotentan and dapagliflozin is administered.

The present disclosure also provides methods of slowing an increase in a nonalcoholic fatty liver disease (NAFLD) fibrosis score (NFS) in a human patient who has chronic kidney disease (CKD) and liver fibrosis, the method comprising administering to the patient a fixed-dose combination of zibotentan and dapagliflozin in an amount effective to reduce the patient's NFS score relative to a dosing regimen in which the patient receives dapagliflozin alone. The present disclosure also provides methods of reducing a urine albumin-creatinine ratio (UACR) and slowing an increase in a nonalcoholic fatty liver disease (NAFLD) fibrosis score (NFS) in a human patient who has chronic kidney disease (CKD) and liver fibrosis, the method comprising administering to the patient a fixed-dose combination of zibotentan and dapagliflozin in an amount effective to reduce the patient's NFS score and to reduce the patient's UACR relative to a dosing regimen in which the patient receives dapagliflozin alone.

The present disclosure also provides a fixed-dose combination of zibotentan and dapagliflozin for use in slowing nonalcoholic fatty liver disease (NAFLD) or cirrhosis in a human patient having CKD and liver fibrosis. In at least embodiment, slowing progression of NAFLD or cirrhosis can be measured by a reduction in fibrosis score (NFS). The present disclosure also provides a fixed-dose combination of zibotentan and dapagliflozin for use in reducing a urine albumin-creatinine ratio (UACR) and slowing an increase in a nonalcoholic fatty liver disease (NAFLD) fibrosis score (NFS) in a human patient having CKD and liver fibrosis.

The present disclosure also provides the use of zibotentan and dapagliflozin in the manufacture of a medicament for slowing an increase in a nonalcoholic fatty liver disease (NAFLD) fibrosis score (NFS) in a human patient having CKD and liver fibrosis, wherein a fixed-dose combination of zibotentan and dapagliflozin is administered. The present disclosure also provides the use of zibotentan and dapagliflozin in the manufacture of a medicament for reducing a urine albumin-creatinine ratio (UACR) and slowing an increase in a nonalcoholic fatty liver disease (NAFLD) fibrosis score (NFS) in a human patient having CKD and liver fibrosis, wherein a fixed-dose combination of zibotentan and dapagliflozin is administered.

The present disclosure also provides a method of reducing levels of one or more inflammatory mediator and/or fibrosis mediator in a human patient who has chronic kidney disease (CKD), the method comprising administering to the patient a fixed-dose combination of zibotentan and dapagliflozin in an amount effective to reduce the patient's levels of one or more inflammatory mediator and/or fibrosis mediator relative to a dosing regimen in which the patient receives dapagliflozin alone. The present disclosure also provides a method of reducing levels of one or more inflammatory mediator and/or fibrosis mediator in a human patient who has chronic kidney disease (CKD) and liver fibrosis, the method comprising administering to the patient a fixed-dose combination of zibotentan and dapagliflozin in an amount effective to reduce the patient's levels of one or more inflammatory mediator and/or fibrosis mediator relative to a dosing regimen in which the patient receives dapagliflozin alone. In some embodiments, the inflammatory mediator and/or fibrosis mediator is one or more of MCP-1, IL-6, and high-sensitivity C-reactive protein (hsCRP).

The present disclosure also provides a fixed-dose combination of zibotentan and dapagliflozin for use in reducing levels of one or more inflammatory mediator and/or fibrosis mediator in a human patient who has chronic kidney disease (CKD), wherein a fixed-dose combination of zibotentan and dapagliflozin is administered. The present disclosure also provides a fixed-dose combination of zibotentan and dapagliflozin for use in reducing levels of one or more inflammatory mediator and fibrosis mediator in a human patient who has chronic kidney disease (CKD) and liver fibrosis, wherein a fixed-dose combination of zibotentan and dapagliflozin is administered. In some embodiments, the inflammatory mediator and/or fibrosis mediator is one or more of MCP-1, IL-6, and high-sensitivity C-reactive protein (hsCRP).

The present disclosure also provides the use of zibotentan and dapagliflozin in the manufacture of a medicament for use in reducing levels of one or more inflammatory mediator and/or fibrosis mediator in a human patient who has chronic kidney disease (CKD), wherein a fixed-dose combination of zibotentan and dapagliflozin is administered. The present disclosure also provides the use of zibotentan and dapagliflozin in the manufacture of a medicament for use in reducing levels of one or more inflammatory mediator and/or fibrosis mediator in a human patient who has chronic kidney disease (CKD) and liver fibrosis, wherein a fixed-dose combination of zibotentan and dapagliflozin is administered. In some embodiments, the inflammatory mediator and/or fibrosis mediator is one or more of MCP-1, IL-6, and high-sensitivity C-reactive protein (hsCRP).

The present disclosure also provides a method of reducing insulin resistance in a human patient who has chronic kidney disease (CKD), the method comprising administering to the patient a fixed-dose combination of zibotentan and dapagliflozin in an amount effective to reduce the patient's insulin resistance relative to a dosing regimen in which the patient receives dapagliflozin alone. The present disclosure also provides a method of reducing insulin resistance in a human patient who has chronic kidney disease (CKD) and liver fibrosis, the method comprising administering to the patient a fixed-dose combination of zibotentan and dapagliflozin in an amount effective to reduce the patient's insulin resistance relative to a dosing regimen in which the patient receives dapagliflozin alone. In some embodiments, the reduction in insulin resistance is shown by assessment of Homeostatic Model Assessment version 2-Insulin Resistance (HOMA2-IR).

The present disclosure also provides a fixed-dose combination of zibotentan and dapagliflozin for use in reducing insulin resistance in a human patient who has chronic kidney disease (CKD), wherein a fixed-dose combination of zibotentan and dapagliflozin is administered. The present disclosure also provides a fixed-dose combination of zibotentan and dapagliflozin for use in reducing insulin resistance in a human patient who has chronic kidney disease (CKD) and liver fibrosis, wherein a fixed-dose combination of zibotentan and dapagliflozin is administered. In some embodiments, the reduction in insulin resistance is shown by assessment of Homeostatic Model Assessment version 2-Insulin Resistance (HOMA2-IR).

The present disclosure also provides the use of zibotentan and dapagliflozin in the manufacture of a medicament for use in reducing insulin resistance in a human patient who has chronic kidney disease (CKD), wherein a fixed-dose combination of zibotentan and dapagliflozin is administered. The present disclosure also provides the use of zibotentan and dapagliflozin in the manufacture of a medicament for use in reducing insulin resistance in a human patient who has chronic kidney disease (CKD) and liver fibrosis, wherein a fixed-dose combination of zibotentan and dapagliflozin is administered. In some embodiments, the reduction in insulin resistance is shown by assessment of Homeostatic Model Assessment version 2-Insulin Resistance (HOMA2-IR).

The present disclosure also provides a method of reducing levels of one or more lipid biomarkers in a human patient who has chronic kidney disease (CKD), the method comprising administering to the patient a fixed-dose combination of zibotentan and dapagliflozin in an amount effective to reduce the patient's levels of one or more lipid biomarker relative to a dosing regimen in which the patient receives dapagliflozin alone. The present disclosure also provides a method of reducing levels of one or more lipid biomarker in a human patient who has chronic kidney disease (CKD) and liver fibrosis, the method comprising administering to the patient a fixed-dose combination of zibotentan and dapagliflozin in an amount effective to reduce the patient's levels of one or more lipid biomarker relative to a dosing regimen in which the patient receives dapagliflozin alone. In some embodiments, the lipid biomarker is one or more of proprotein convertase subtilisin/kexin type 9 (PCSK9), lipoprotein (a) (Lp(a)), low-density lipoprotein cholesterol (LDL-C), apolipoprotein B (ApoB). In some embodiments, the method increases the LDL-C/ApoB ratio relative to a dosing regimen in which the patient receives dapagliflozin alone.

The present disclosure also provides a fixed-dose combination of zibotentan and dapagliflozin for use in reducing levels of one or more lipid biomarkers in a human patient who has chronic kidney disease (CKD), wherein a fixed-dose combination of zibotentan and dapagliflozin is administered. The present disclosure also provides a fixed-dose combination of zibotentan and dapagliflozin for use in reducing levels of one or more lipid biomarker in a human patient who has chronic kidney disease (CKD) and liver fibrosis, wherein a fixed-dose combination of zibotentan and dapagliflozin is administered. In some embodiments, the lipid biomarker is one or more of proprotein convertase subtilisin/kexin type 9 (PCSK9), lipoprotein (a) (Lp(a)), low-density lipoprotein cholesterol (LDL-C), apolipoprotein B (ApoB). In some embodiments, the use increases the LDL-C/ApoB ratio relative to a dosing regimen in which the patient receives dapagliflozin alone.

The present disclosure also provides the use of zibotentan and dapagliflozin in the manufacture of a medicament for use in reducing levels of one or more lipid biomarkers in a human patient who has chronic kidney disease (CKD), wherein a fixed-dose combination of zibotentan and dapagliflozin is administered. The present disclosure also provides the use of zibotentan and dapagliflozin in the manufacture of a medicament for use in reducing levels of one or more lipid biomarker in a human patient who has chronic kidney disease (CKD) and liver fibrosis, wherein a fixed-dose combination of zibotentan and dapagliflozin is administered. In some embodiments, the lipid biomarker is one or more of proprotein convertase subtilisin/kexin type 9 (PCSK9), lipoprotein (a) (Lp(a)), low-density lipoprotein cholesterol (LDL-C), apolipoprotein B (ApoB). In some embodiments, the use increases the LDL-C/ApoB ratio relative to a dosing regimen in which the patient receives dapagliflozin alone.

In some embodiments, the patient is naïve to a sodium-glucose cotransporter-2 (SGLT2) inhibitor. In some embodiments, the patient is not naïve to a SGLT2 inhibitor. In some embodiments, the patient has an estimated glomerular filtration rate (eGFR) of 20-90 mL/min/1.73 m². In some embodiments, the fixed-dose combination of zibotentan and dapagliflozin is administered to the patient once per day.

In some embodiments, the methods described herein comprise administering zibotentan at a dose of 0.25 mg to 5 mg. In some embodiments, the methods described herein comprise administering zibotentan at a dose of 0.25 mg. In some embodiments, the methods described herein comprise administering zibotentan at a dose of 0.5 mg. In some embodiments, the methods described herein comprise administering zibotentan at a dose of 0.75 mg. In some embodiments, the methods described herein comprise administering zibotentan at a dose of 1.0 mg. In some embodiments, the methods described herein comprise administering zibotentan at a dose of 1.25 mg. In some embodiments, the methods described herein comprise administering zibotentan at a dose of 1.5 mg. In some embodiments, the methods described herein comprise administering zibotentan at a dose of 5 mg. In some embodiments, the methods described herein comprise administering dapagliflozin at a dose of 2.5 mg to 10 mg. In some embodiments, the methods described herein comprise administering dapagliflozin at a dose of 2.5 mg. In some embodiments, the methods described herein comprise administering dapagliflozin at a dose of 5.0 mg. In some embodiments, the methods described herein comprise administering dapagliflozin at a dose of 10.0 mg.

In some embodiments, the methods described herein comprise administering zibotentan at a dose of 5 mg and dapagliflozin at a dose of 10 mg. In some embodiments, the methods described herein comprise administering zibotentan at a dose of 1.5 mg and dapagliflozin at a dose of 10 mg. In some embodiments, the methods described herein comprise administering zibotentan at a dose of 0.25 mg and dapagliflozin at a dose of 10 mg.

In some embodiments, the methods described herein comprise administering zibotentan at a dose of 0.25 mg and dapagliflozin at a dose of 10 mg when the patient has an eGFR that is <45 mL/min/1.73 m², and administering zibotentan at a dose of 1.5 mg and dapagliflozin at a dose of 10 mg when the patient has an eGFR that is ≥45 mL/min/1.73 m². In some embodiments, the fixed dose combination of zibotentan and dapagliflozin is adjusted from 1.5 mg of zibotentan and 10 mg of dapagliflozin to 0.25 mg of zibotentan and 10 mg of dapagliflozin when the patient's eGFR changes to <45 mL/min/1.73 m². In some embodiments, the fixed dose combination of zibotentan and dapagliflozin is adjusted from 0.25 mg of zibotentan and 10 mg of dapagliflozin to 1.5 mg of zibotentan and 10 mg of dapagliflozin when the patient's eGFR changes to ≥45 mL/min/1.73 m².

In some embodiments, administration of the fixed-dose combination of zibotentan and dapagliflozin reduces the patient's NFS to below 0.675. In some embodiments, the methods described herein result in a partial remission or remission of the patient's liver fibrosis. In some embodiments, administration of the fixed-dose combination of zibotentan and dapagliflozin does not increase the patient's NFS relative to a dosing regimen in which the patient receives dapagliflozin alone. In some embodiments, administration of the fixed-dose combination of zibotentan and dapagliflozin reduces the risk of increased NFS in the patient relative to a dosing regimen in which the patient receives dapagliflozin alone.

In some embodiments, administration of the fixed-dose combination of zibotentan and dapagliflozin reduces the patient's liver fibrosis relative to a dosing regimen in which the patient receives dapagliflozin alone. In some embodiments, administration of the fixed-dose combination of zibotentan and dapagliflozin reduces the patient's liver fibrosis and reduces the patient's UACR relative to a dosing regimen in which the patient receives dapagliflozin alone.

In some embodiments, administration of the fixed-dose combination of zibotentan and dapagliflozin increases the patient's platelet count, serum albumin, or both platelet count and serum albumin. In some embodiments, administration of the fixed-dose combination of zibotentan and dapagliflozin reduces the patient's aspartate aminotransferase (AST) level, alanine aminotransferase (ALT) level, or both AST and ALT levels.

### BRIEF DESCRIPTION OF THE DRAWINGS

FIG. 1 shows the impact of a combination of zibotentan and dapagliflozin (0.25 mg and 10 mg, respectively; 1.5 mg and 10 mg, respectively) relative to dapagliflozin (10 mg) alone on urine albumin-creatinine ratio (UACR) in patients in the ZENITH-CKD study having an NFS>0.675.

### DETAILED DESCRIPTION

As used herein, the terms “about” and “approximately,” when used to modify a numeric value or numeric range, indicate that deviations of up to 10% above and down to 10% below the value or range remain within the intended meaning of the recited value or range. In some embodiments, “about” refers to ±10%. In some embodiments, “about” refers to ±9%. In some embodiments, “about” refers to ±8%. In some embodiments, “about” refers to ±7%. In some embodiments, “about” refers to ±6%. In some embodiments, “about” refers to ±5%. In some embodiments, “about” refers to ±4%. In some embodiments, “about” refers to ±3%. In some embodiments, “about” refers to ±2%. In some embodiments, “about” refers to ±1%. It is understood that wherever aspects are described herein with the language “about” or “approximately” a numeric value or range, otherwise analogous aspects referring to the specific numeric value or range (without “about”) are also provided. It is also understood that wherever aspects are described herein referring to a numeric value or range without the language “about” or “approximately,” otherwise analogous aspects referring to “about” or “approximately” the specific numeric value or range are also provided.

The terms “treating” or “treatment” or “to treat” refer to therapeutic measures that cure, slow down, lessen symptoms of, and/or halt progression of a diagnosed pathologic disease, disorder, or condition. Treatment need not result in a complete cure of the condition; partial inhibition or reduction of the condition being treated is encompassed by this term.

The effectiveness of the compounds of the present disclosure in treating and/or preventing chronic kidney disease (CKD) and liver fibrosis and/or diseases, disorders, and/or conditions associated therewith can readily be determined by a person of ordinary skill in the relevant art. Determining and adjusting an appropriate dosing regimen (e.g., adjusting the amount of compound per dose and/or number of doses and frequency of dosing) can also readily be performed by a person of ordinary skill in the relevant art. One or any combination of diagnostic methods, including physical examination, assessment and monitoring of clinical symptoms, and performance of analytical tests and methods described herein, may be used for monitoring the health status of the patient.

An “effective amount” or “therapeutically effective amount” refers to an amount of at least one compound of the present disclosure or a pharmaceutical composition comprising at least one such compound that, when administered to a patient, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the patient. Patients may generally be monitored for therapeutic effectiveness using assays suitable for the disease, disorder, and/or condition being treated or prevented, which assays will be familiar to those having ordinary skill in the art, such as those described herein. The level of a compound that is administered to a patient may be monitored by determining the level of the compound (or a metabolite of the compound) in a biological fluid, for example, in the blood, blood fraction (e.g., serum), and/or in the urine, and/or another biological sample from the patient. Any method practiced in the art to detect the compound, or metabolite thereof, may be used to measure the level of the compound during the course of a therapeutic regimen.

The dose of a compound described herein may depend upon the patient's condition, that is, stage of the disease, severity of symptoms caused by the disease, general health status, as well as age, gender, and weight, and other factors apparent to a person of ordinary skill in the medical art. Similarly, the dose of the therapeutic for treating a disease, disorder, and/or condition may be determined according to parameters understood by a person of ordinary skill in the medical art.

As used herein, the terms “subject” and “patient” are used interchangeably to refer to a party receiving a medical treatment. In some aspects, the subject is a human.

As used herein, the term “albuminuria” refers to the presence of albumin in urine, a marker for chronic kidney disease. Albuminuria is present when the urine albumin to creatine ratio (UACR) is above 30 mg/g.

As used herein, the term “proteinuria” refers to the presence of protein in urine, a marker for chronic kidney disease. Proteinuria is present when the urine protein to creatine ratio (UPCR) is above 150 mg/g. In some embodiments, proteinuria is present when the UPCR is above 200 mg/g.

As used herein, the “severity” of chronic kidney disease (CKD) refers to the stage or grade of CKD as defined by the Kidney Disease Improving Global Outcomes (KIDGO) Guidelines. Stage 1 is normal or high eGFR (eGFR>90 mL/min/1.73 m²). Stage 2 is mild CKD (eGFR=60-89 mL/min/1.73 m²). Stage 3A is moderate CKD (eGFR=45-59 mL/min/1.73 m²). Stage 3B is moderate CKD (eGFR=30-44 mL/min/1.73 m²). Stage 4 is severe CKD (eGFR=15-29 mL/min/1.73 m²). Stage 5 is end-stage CKD (eGFR<15 mL/min/1.73 m²).

As used herein, the term “end-stage kidney disease (ESKD)” refers to (i) having a sustained eGFR<15 mL/min/1.73 m², (ii) receiving chronic dialysis treatment, or (iii) receiving a renal transplant. In some embodiments, “sustained” refers to a confirmation of a similar eGFR measurement by a second eGFR test 3 months apart.

As used herein, the term “NFS” refers to a composite score that correlates to the amount of scarring in the liver based on a number of parameters and laboratory tests (Angulo P et al., The NAFLD fibrosis score: a noninvasive system that identifies liver fibrosis in patients with NAFLD. Hepatology. 2007; 45:846-854). The NFS calculation accounts for the subject's age, body mass index, hyperglycemia, platelet count, albumin level, and aspartate aminotransferase level/alanine aminotransferase level (AST/ALT) ratio. A NFS of greater than 0.675 indicates a high risk for advanced fibrosis.

As used herein, a patient is “suspected” of having liver fibrosis when the patient has an NFS of >0.675, indicating a high likelihood of advanced fibrosis.

As used herein, the phrase “not naive to SGLT2 inhibitors” refers to a patient who has (i) previously received SGLT2 inhibitor therapy; and/or (ii) is currently receiving SGLT2 inhibitor therapy.

The terms “administer,” “administering,” “administration,” and the like, as used herein, refer to methods that may be used to enable delivery of a drug, e.g., zibotentan or a pharmaceutically acceptable salt, solvate, mixed solvate, complex, or prodrug thereof and dapagliflozin or a pharmaceutically acceptable salt, solvate, mixed solvate, complex, or prodrug thereof, as described herein. Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa. In some aspects, zibotentan and dapagliflozin are administered orally.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed, is appropriate for the formulation employed, and is compatible with other ingredients of the formulation.

A “sterile” formulation is aseptic or essentially free from living microorganisms and their spores.

As used herein, the term “prodrug” refers to, for example, esters and carbonates that may be converted, for example, under physiological conditions or by solvolysis, to zibotentan or dapagliflozin. Thus, the term prodrug includes metabolic precursors of zibotentan or dapagliflozin that are pharmaceutically acceptable. The term prodrug also includes covalently bonded carriers that release zibotentan or dapagliflozin in vivo when such prodrug is administered to a patient. Non-limiting examples of prodrugs include esters and carbonates.

Various forms of prodrugs are known in the art. For examples of such prodrug derivatives, see: (1) Design of Prodrugs, edited by H. Bundgaard, (Elsevier, 1985) and Methods in Enzymology, Vol. 42, p. 309-396, edited by K. Widder, et al. (Academic Press, 1985); (2) A Textbook of Drug Design and Development, edited by Krogsgaard-Larsen and H. Bundgaard, Chapter 5 “Design and Application of Prodrugs”, by H. Bundgaard p. 113-191 (1991); (3) H. Bundgaard, Advanced Drug Delivery Reviews, 8, 1-38 (1992); (4) H. Bundgaard, et al., Journal of Pharmaceutical Sciences, 77, 285 (1988); and (5) N. Kakeya, et al., Chem Pharm Bull, 32, 692 (1984).

It is understood that wherever aspects are described herein with the language “comprising,” otherwise analogous aspects described in terms of “consisting of” and/or “consisting essentially of” are also provided. In this disclosure, “comprises,” “comprising,” “containing,” and “having” and the like can mean “includes,” “including,” and the like; “consisting essentially of” or “consists essentially” are open-ended, allowing for the presence of more than that which is recited so long as basic or novel characteristics as described herein of that which is recited are not changed by the presence of more than that which is recited, but excludes prior art aspects.

Unless specifically stated or obvious from context, as used herein, the term “or” is understood to be inclusive.

In an aspect, the disclosure herein provides methods of treating CKD and liver fibrosis in a human patient comprising administering a fixed-dose combination of zibotentan and dapagliflozin to a patient in need thereof.

In an aspect, the disclosure herein provides a fixed dose combination of zibotentan and dapagliflozin for use in treating CKD and liver fibrosis in a human patient.

In an aspect, the disclosure herein provides the use of zibotentan and dapagliflozin in the manufacture of a medicament for the treatment of CKD and liver fibrosis in a human patient, wherein a fixed-dose combination of zibotentan and dapagliflozin is administered.

In an aspect, the disclosure herein provides methods of slowing liver fibrosis in a human patient having CKD and liver fibrosis, comprising administering a fixed-dose combination of zibotentan and dapagliflozin to a patient in need thereof. In an aspect, the disclosure herein provides methods of reducing a patient's UACR and slowing liver fibrosis in a human patient having CKD and liver fibrosis, comprising administering a fixed-dose combination of zibotentan and dapagliflozin to a patient in need thereof.

In an aspect, the disclosure herein provides a fixed-dose combination of zibotentan and dapagliflozin for use in slowing liver fibrosis in a human patient having CKD and liver fibrosis. In an aspect, the disclosure herein provides a fixed-dose combination of zibotentan and dapagliflozin for use in reducing a patient's UACR and slowing liver fibrosis in a human patient having CKD and liver fibrosis.

In an aspect, the disclosure herein provides the use of zibotentan and dapagliflozin in the manufacture of a medicament for slowing liver fibrosis in a human patient having CKD and liver fibrosis, wherein a fixed-dose combination of zibotentan and dapagliflozin is administered. In an aspect, the disclosure herein provides the use of zibotentan and dapagliflozin in the manufacture of a medicament for reducing a patient's UACR and slowing liver fibrosis in a human patient having CKD and liver fibrosis, wherein a fixed-dose combination of zibotentan and dapagliflozin is administered.

In an aspect, the disclosure herein provides methods of reducing liver fibrosis in a human patient having CKD and liver fibrosis, comprising administering a fixed-dose combination of zibotentan and dapagliflozin to a patient in need thereof. In an aspect, the disclosure herein provides methods of reducing a patient's UACR and liver fibrosis in a human patient having CKD and liver fibrosis, comprising administering a fixed-dose combination of zibotentan and dapagliflozin to a patient in need thereof.

In an aspect, the disclosure herein provides a fixed-dose combination of zibotentan and dapagliflozin for use in reducing liver fibrosis in a human patient having CKD and liver fibrosis. In an aspect, the disclosure herein provides a fixed-dose combination of zibotentan and dapagliflozin for use in reducing a patient's UACR and liver fibrosis in a human patient having CKD and liver fibrosis.

In an aspect, the disclosure herein provides the use of zibotentan and dapagliflozin in the manufacture of a medicament for reducing liver fibrosis in a human patient having CKD and liver fibrosis, wherein a fixed-dose combination of zibotentan and dapagliflozin is administered. In an aspect, the disclosure herein provides the use of zibotentan and dapagliflozin in the manufacture of a medicament for reducing a patient's UACR and liver fibrosis in a human patient having CKD and liver fibrosis, wherein a fixed-dose combination of zibotentan and dapagliflozin is administered.

In an aspect, the disclosure herein also provides methods of slowing nonalcoholic fatty liver disease (NAFLD) or cirrhosis in a human patient who has chronic kidney disease (CKD) and liver fibrosis, the method comprising administering to the patient a fixed-dose combination of zibotentan and dapagliflozin in an amount effective to slow the patient's NAFLD or cirrhosis relative to a dosing regimen in which the patient receives dapagliflozin alone. In at least one embodiment, slowing NAFLD or cirrhosis can be measured by a reduction in fibrosis score (NFS).

In an aspect, the disclosure herein also provides a fixed-dose combination of zibotentan and dapagliflozin for use in slowing nonalcoholic fatty liver disease (NAFLD) or cirrhosis in a human patient having CKD and liver fibrosis. In at least one embodiment, slowing NAFLD or cirrhosis can be measured by a reduction in fibrosis score (NFS).

In an aspect, the disclosure herein also provides the use of zibotentan and dapagliflozin in the manufacture of a medicament for slowing nonalcoholic fatty liver disease (NAFLD) or cirrhosis in a human patient having CKD and liver fibrosis, wherein a fixed-dose combination of zibotentan and dapagliflozin is administered. In at least one embodiment, slowing NAFLD or cirrhosis can be measured by a reduction in fibrosis score (NFS).

In an aspect, the disclosure herein provides methods of reducing a patient's non-alcoholic fatty liver (NAFLD) fibrosis score (NFS) wherein the patient has CKD and liver fibrosis, the methods comprising administering a fixed-dose combination of zibotentan and dapagliflozin to a patient in need thereof. In an aspect, the disclosure herein provides methods of reducing a patient's UACR and a patient's non-alcoholic fatty liver (NAFLD) fibrosis score (NFS) wherein the patient has CKD and liver fibrosis, the methods comprising administering a fixed-dose combination of zibotentan and dapagliflozin to a patient in need thereof.

In an aspect, the disclosure herein provides a fixed-dose combination of zibotentan and dapagliflozin for use in reducing a patient's NFS, wherein the patient has CKD and liver fibrosis and is in need thereof. In an aspect, the disclosure herein provides a fixed-dose combination of zibotentan and dapagliflozin for use in reducing a patient's UACR and a patient's NFS, wherein the patient has CKD and liver fibrosis and is in need thereof.

In an aspect, the disclosure herein provides the use of zibotentan and dapagliflozin in the manufacture of a medicament for reducing a patient's NFS, wherein the patient has CKD and liver fibrosis and is in need thereof, wherein a fixed-dose combination of zibotentan and dapagliflozin is administered. In an aspect, the disclosure herein provides the use of zibotentan and dapagliflozin in the manufacture of a medicament for reducing a patient's UACR and a patient's NFS, wherein the patient has CKD and liver fibrosis and is in need thereof, wherein a fixed-dose combination of zibotentan and dapagliflozin is administered.

In an aspect, the disclosure herein provides methods of slowing an increase in a patient's non-alcoholic fatty liver (NAFLD) fibrosis score (NFS) wherein the patient has CKD and liver fibrosis, the methods comprising administering a fixed-dose combination of zibotentan and dapagliflozin to a patient in need thereof. In an aspect, the disclosure herein provides methods of reducing a patient's UACR and slowing an increase in a patient's non-alcoholic fatty liver (NAFLD) fibrosis score (NFS) wherein the patient has CKD and liver fibrosis, the methods comprising administering a fixed-dose combination of zibotentan and dapagliflozin to a patient in need thereof.

In an aspect, the disclosure herein provides a fixed-dose combination of zibotentan and dapagliflozin for use in slowing an increase in a patient's NFS, wherein the patient has CKD and liver fibrosis and is in need thereof. In an aspect, the disclosure herein provides a fixed-dose combination of zibotentan and dapagliflozin for use in reducing a patient's UACR and slowing an increase in a patient's NFS, wherein the patient has CKD and liver fibrosis and is in need thereof.

In an aspect, the disclosure herein provides the use of zibotentan and dapagliflozin in the manufacture of a medicament for slowing an increase in a patient's NFS, wherein the patient has CKD and liver fibrosis and is in need thereof, wherein a fixed-dose combination of zibotentan and dapagliflozin are administered. In an aspect, the disclosure herein provides the use of zibotentan and dapagliflozin in the manufacture of a medicament for reducing a patient's UACR and slowing an increase in a patient's NFS, wherein the patient has CKD and liver fibrosis and is in need thereof, wherein a fixed-dose combination of zibotentan and dapagliflozin are administered.

In an aspect, the disclosure herein provides methods of increasing a patient's platelet count, serum albumin, or both platelet count and serum albumin, wherein the patient has CKD and liver fibrosis, the methods comprising administering a fixed-dose combination of zibotentan and dapagliflozin to a patient in need thereof.

In an aspect, the disclosure herein provides a fixed-dose combination of zibotentan and dapagliflozin for use in increasing a patient's platelet count, serum albumin, or both platelet count and serum albumin, wherein the patient has CKD and liver fibrosis and is in need thereof.

In an aspect, the disclosure herein provides the use of zibotentan and dapagliflozin in the manufacture of a medicament for increasing a patient's platelet count, serum albumin, or both platelet count and serum albumin, wherein the patient has CKD and liver fibrosis and is in need thereof, wherein a fixed-dose combination of zibotentan and dapagliflozin are administered.

In an aspect, the disclosure herein provides methods of reducing a patient's aspartate aminotransferase (AST) level, alanine aminotransferase (ALT) level, or both AST and ALT levels, wherein the patient has CKD and liver fibrosis, the methods comprising administering a fixed-dose combination of zibotentan and dapagliflozin to a patient in need thereof.

In an aspect, the disclosure herein provides a fixed-dose combination of zibotentan and dapagliflozin for use in reducing a patient's aspartate aminotransferase (AST) level, alanine aminotransferase (ALT) level, or both AST and ALT levels, wherein the patient has CKD and liver fibrosis and is in need thereof.

In an aspect, the disclosure herein provides the use of zibotentan and dapagliflozin in the manufacture of a medicament for reducing a patient's aspartate aminotransferase (AST) level, alanine aminotransferase (ALT) level, or both AST and ALT levels, wherein the patient has CKD and liver fibrosis and is in need thereof, wherein a fixed-dose combination of zibotentan and dapagliflozin are administered.

In an aspect, the disclosure herein provides a method of reducing levels of one or more inflammatory mediator and/or fibrosis mediator in a human patient who has chronic kidney disease (CKD), the method comprising administering to the patient a fixed-dose combination of zibotentan and dapagliflozin in an amount effective to reduce the patient's levels of one or more inflammatory mediator and/or fibrosis mediator relative to a dosing regimen in which the patient receives dapagliflozin alone. In an aspect, the disclosure herein provides a method of reducing levels of one or more inflammatory mediator and/or fibrosis mediator in a human patient who has chronic kidney disease (CKD) and liver fibrosis, the method comprising administering to the patient a fixed-dose combination of zibotentan and dapagliflozin in an amount effective to reduce the patient's levels of one or more inflammatory mediator and/or fibrosis mediator relative to a dosing regimen in which the patient receives dapagliflozin alone. In some embodiments, the inflammatory mediator and/or fibrosis mediator is one or more of MCP-1, IL-6, and high-sensitivity C-reactive protein (hsCRP). In some embodiments, levels of each of MCP-1, IL-6, and high-sensitivity C-reactive protein (hsCRP) are reduced.

In an aspect, the disclosure herein provides a fixed-dose combination of zibotentan and dapagliflozin for use in reducing levels of one or more inflammatory mediator and/or fibrosis mediator in a human patient who has chronic kidney disease (CKD), wherein a fixed-dose combination of zibotentan and dapagliflozin is administered. In an aspect, the disclosure herein provides a fixed-dose combination of zibotentan and dapagliflozin for use in reducing levels of one or more inflammatory mediator and fibrosis mediator in a human patient who has chronic kidney disease (CKD) and liver fibrosis, wherein a fixed-dose combination of zibotentan and dapagliflozin is administered. In some embodiments, the inflammatory mediator and/or fibrosis mediator is one or more of MCP-1, IL-6, and high-sensitivity C-reactive protein (hsCRP). In some embodiments, levels of each of MCP-1, IL-6, and high-sensitivity C-reactive protein (hsCRP) are reduced.

In an aspect, the disclosure herein provides the use of zibotentan and dapagliflozin in the manufacture of a medicament for use in reducing levels of one or more inflammatory mediator and/or fibrosis mediator in a human patient who has chronic kidney disease (CKD), wherein a fixed-dose combination of zibotentan and dapagliflozin is administered. In an aspect, the disclosure herein provides the use of zibotentan and dapagliflozin in the manufacture of a medicament for use in reducing levels of one or more inflammatory mediator and/or fibrosis mediator in a human patient who has chronic kidney disease (CKD) and liver fibrosis, wherein a fixed-dose combination of zibotentan and dapagliflozin is administered. In some embodiments, the inflammatory mediator and/or fibrosis mediator is one or more of MCP-1, IL-6, and high-sensitivity C-reactive protein (hsCRP). In some embodiments, levels of each of MCP-1, IL-6, and high-sensitivity C-reactive protein (hsCRP) are reduced.

In an aspect, the disclosure herein provides a method of reducing insulin resistance in a human patient who has chronic kidney disease (CKD), the method comprising administering to the patient a fixed-dose combination of zibotentan and dapagliflozin in an amount effective to reduce the patient's insulin resistance relative to a dosing regimen in which the patient receives dapagliflozin alone. In an aspect, the disclosure herein provides a method of reducing insulin resistance in a human patient who has chronic kidney disease (CKD) and liver fibrosis, the method comprising administering to the patient a fixed-dose combination of zibotentan and dapagliflozin in an amount effective to reduce the patient's insulin resistance relative to a dosing regimen in which the patient receives dapagliflozin alone. In some embodiments, the reduction in insulin resistance is shown by assessment of Homeostatic Model Assessment version 2—Insulin Resistance (HOMA2-IR).

In an aspect, the disclosure herein provides a fixed-dose combination of zibotentan and dapagliflozin for use in reducing insulin resistance in a human patient who has chronic kidney disease (CKD), wherein a fixed-dose combination of zibotentan and dapagliflozin is administered. In an aspect, the disclosure herein provides a fixed-dose combination of zibotentan and dapagliflozin for use in reducing insulin resistance in a human patient who has chronic kidney disease (CKD) and liver fibrosis, wherein a fixed-dose combination of zibotentan and dapagliflozin is administered. In some embodiments, the reduction in insulin resistance is shown by assessment of Homeostatic Model Assessment version 2-Insulin Resistance (HOMA2-IR).

In an aspect, the disclosure herein provides the use of zibotentan and dapagliflozin in the manufacture of a medicament for use in reducing insulin resistance in a human patient who has chronic kidney disease (CKD), wherein a fixed-dose combination of zibotentan and dapagliflozin is administered. In an aspect, the disclosure herein provides the use of zibotentan and dapagliflozin in the manufacture of a medicament for use in reducing insulin resistance in a human patient who has chronic kidney disease (CKD) and liver fibrosis, wherein a fixed-dose combination of zibotentan and dapagliflozin is administered. In some embodiments, the reduction in insulin resistance is shown by assessment of Homeostatic Model Assessment version 2-Insulin Resistance (HOMA2-IR).

In an aspect, the disclosure herein provides a method of reducing levels of one or more lipid biomarkers in a human patient who has chronic kidney disease (CKD), the method comprising administering to the patient a fixed-dose combination of zibotentan and dapagliflozin in an amount effective to reduce the patient's levels of one or more lipid biomarker relative to a dosing regimen in which the patient receives dapagliflozin alone. In an aspect, the disclosure herein provides a method of reducing levels of one or more lipid biomarker in a human patient who has chronic kidney disease (CKD) and liver fibrosis, the method comprising administering to the patient a fixed-dose combination of zibotentan and dapagliflozin in an amount effective to reduce the patient's levels of one or more lipid biomarker relative to a dosing regimen in which the patient receives dapagliflozin alone. In some embodiments, the lipid biomarker is one or more of proprotein convertase subtilisin/kexin type 9 (PCSK9), lipoprotein (a) (Lp(a)), low-density lipoprotein cholesterol (LDL-C), apolipoprotein B (ApoB). In some embodiments, levels of each of PCSK9, Lp(a), LDL-C, and ApoB are each reduced. In some embodiments, the method increases the LDL-C/ApoB ratio relative to a dosing regimen in which the patient receives dapagliflozin alone.

In an aspect, the disclosure herein provides a fixed-dose combination of zibotentan and dapagliflozin for use in reducing levels of one or more lipid biomarkers in a human patient who has chronic kidney disease (CKD), wherein a fixed-dose combination of zibotentan and dapagliflozin is administered. In an aspect, the disclosure herein provides a fixed-dose combination of zibotentan and dapagliflozin for use in reducing levels of one or more lipid biomarker in a human patient who has chronic kidney disease (CKD) and liver fibrosis, wherein a fixed-dose combination of zibotentan and dapagliflozin is administered. In some embodiments, the lipid biomarker is one or more of proprotein convertase subtilisin/kexin type 9 (PCSK9), lipoprotein (a) (Lp(a)), low-density lipoprotein cholesterol (LDL-C), apolipoprotein B (ApoB). In some embodiments, levels of each of PCSK9, Lp(a), LDL-C, and ApoB are each reduced. In some embodiments, the use increases the LDL-C/ApoB ratio relative to a dosing regimen in which the patient receives dapagliflozin alone.

In an aspect, the disclosure herein provides the use of zibotentan and dapagliflozin in the manufacture of a medicament for use in reducing levels of one or more lipid biomarkers in a human patient who has chronic kidney disease (CKD), wherein a fixed-dose combination of zibotentan and dapagliflozin is administered. In an aspect, the disclosure herein provides the use of zibotentan and dapagliflozin in the manufacture of a medicament for use in reducing levels of one or more lipid biomarker in a human patient who has chronic kidney disease (CKD) and liver fibrosis, wherein a fixed-dose combination of zibotentan and dapagliflozin is administered. In some embodiments, the lipid biomarker is one or more of proprotein convertase subtilisin/kexin type 9 (PCSK9), lipoprotein (a) (Lp(a)), low-density lipoprotein cholesterol (LDL-C), apolipoprotein B (ApoB). In some embodiments, levels of each of PCSK9, Lp(a), LDL-C, and ApoB are each reduced. In some embodiments, the use increases the LDL-C/ApoB ratio relative to a dosing regimen in which the patient receives dapagliflozin alone.

In some embodiments, the chronic kidney disease (CKD) is CKD of stage 1 to 4 as defined by the Kidney Disease Improving Global Outcomes (KIDGO) Guidelines. In some embodiments, the CKD is CKD of stages 2-3. In some embodiments, the CKD is CKD of stages 3-4. In some embodiments, the CKD is CKD of stages 2-4. In some embodiments, the CKD is CKD of stage 4. In some embodiments, the CKD is CKD of stage 3. In some embodiments, the CKD is CKD of stages 3A or 3B. In some embodiments, the CKD is CKD of stage 2. In some embodiments, the CKD is CKD of stage 1.

In some embodiments, the patient is suspected of having liver fibrosis. In some embodiments, the patient has liver fibrosis. In some embodiments, the patient has an NFS of >−1.5 to <0.675. In some embodiments, the patient has a NFS of >0.675.

In some embodiments, the patient is a CKD human patient having liver fibrosis and Type 2 diabetes. In some embodiments, the patient is a CKD human patient having liver fibrosis and without Type 2 diabetes.

In some embodiments, the patient has an estimated glomerular filtration rate (eGFR) of 20-90 mL/min/1.73 m². In some embodiments, the patient has an eGFR of 20-29 mL/min/1.73 m². In some embodiments, the patient has an eGFR of 30-44 mL/min/1.73 m². In some embodiments, the patient has an eGFR of 45-59 mL/min/1.73 m². In some embodiments, the patient has an eGFR of 60-89 mL/min/1.73 m².

In some embodiments, the patient has an eGFR of 20-90 mL/min/1.73 m² and a NFS of >0.675. In some embodiments, the patient has an eGFR of 20-29 mL/min/1.73 m² and a NFS of >0.675. In some embodiments, the patient has an eGFR of 30-44 mL/min/1.73 m² and a NFS of >0.675. In some embodiments, the patient has an eGFR of 45-59 mL/min/1.73 m² and a NFS of >0.675. In some embodiments, the patient has an eGFR of 60-89 mL/min/1.73 m² and a NFS of >0.675.

In some embodiments, the patient is naïve to a sodium-glucose cotransporter 2 (SGLT2) inhibitor. In some embodiments wherein the patient is naïve to a SGLT2 inhibitor, the patient receives dapagliflozin (run-in) prior to receiving the fixed-dose combination of zibotentan and dapagliflozin. In some embodiments wherein the patient is naïve to a SGLT2 inhibitor, the patient receives a 4-week run-in of dapagliflozin prior to receiving the fixed-dose combination of zibotentan and dapagliflozin. In some embodiments, the patient is not naive to a SGLT2 inhibitor.

In some embodiments, the patient is naïve to SGLT2 inhibitors and has an eGFR of 20-90 mL/min/1.73 m² and a NFS of >0.675. In some embodiments, the patient is naïve to SGLT2 inhibitors and has an eGFR of 20-29 mL/min/1.73 m² and a NFS of >0.675. In some embodiments, the patient is naïve to SGLT2 inhibitors and has an eGFR of 30-44 mL/min/1.73 m² and a NFS of >0.675. In some embodiments, the patient is naïve to SGLT2 inhibitors and has an eGFR of 45-59 mL/min/1.73 m² and a NFS of >0.675. In some embodiments, the patient is naïve to SGLT2 inhibitors and has an eGFR of 60-89 mL/min/1.73 m² and a NFS of >0.675.

In some embodiments, the patient is not naïve to SGLT2 inhibitors and has an eGFR of 20-90 mL/min/1.73 m² and a NFS of >0.675. In some embodiments, the patient is not naïve to SGLT2 inhibitors and has an eGFR of 20-29 mL/min/1.73 m² and a NFS of >0.675. In some embodiments, the patient is not naïve to SGLT2 inhibitors and has an eGFR of 30-44 mL/min/1.73 m² and a NFS of >0.675. In some embodiments, the patient is not naïve to SGLT2 inhibitors and has an eGFR of 45-59 mL/min/1.73 m² and a NFS of >0.675. In some embodiments, the patient is not naïve to SGLT2 inhibitors and has an eGFR of 60-89 mL/min/1.73 m² and a NFS of >0.675.

In some embodiments, the patients described herein are administered a fixed-dose combination of zibotentan, or a pharmaceutically acceptable salt thereof, and dapagliflozin, or a pharmaceutically acceptable salt thereof.

In some embodiments, zibotentan, or a pharmaceutically acceptable salt thereof, is administered once daily.

In some embodiments, the total daily dose of zibotentan, or the pharmaceutically acceptable salt thereof, administered is 0.25 mg to 5 mg. In some embodiments, the total daily dose of zibotentan is 0.25 mg. In some embodiments, the total daily dose of zibotentan is 0.5 mg. In some embodiments, the total daily dose of zibotentan is 0.75 mg. In some embodiments, the total daily dose of zibotentan is 1.0 mg. In some embodiments, the total daily dose of zibotentan is 1.25 mg. In some embodiments, the total daily dose of zibotentan is 1.5 mg. In some embodiments, the total daily dose of zibotentan is 5 mg.

In some embodiments, zibotentan, or a pharmaceutically acceptable salt thereof, is in tablet form. In some embodiments, zibotentan, or a pharmaceutically acceptable salt thereof, is administered in the form of a pharmaceutical composition comprising one or more pharmaceutically acceptable excipients. In some embodiments, the composition comprises one or more pharmaceutical diluents, one or more pharmaceutical disintegrants, or one or more pharmaceutical lubricants.

In some embodiments, dapagliflozin, or a pharmaceutically acceptable salt thereof, is administered once daily.

In an embodiment, dapagliflozin is in the form of a pharmaceutically acceptable solvate, mixed solvate, or complex. In some aspects provided herein, dapagliflozin is in the form of a non-crystalline solid. In some aspects provided herein, dapagliflozin is in the form of a crystalline solid. In some aspects provided herein, dapagliflozin is in the form of a(S)-propylene glycol ((S)-PG) solvate which has the structure:

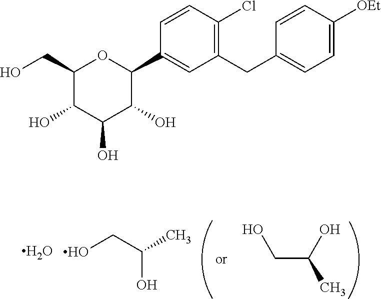

In aspects provided herein, dapagliflozin is administered to the patient orally. In aspects provided herein, dapagliflozin is administered to the patient in a tablet form.

In some embodiments, the total daily dose of dapagliflozin, or the pharmaceutically acceptable salt thereof, administered is 2.5 mg to 10 mg. In some embodiments, the total daily dose of dapagliflozin is 2.5 mg. In some embodiments, the total daily dose of dapagliflozin is 3.0 mg. In some embodiments, the total daily dose of dapagliflozin is 3.5 mg. In some embodiments, the total daily dose of dapagliflozin is 4.0 mg. In some embodiments, the total daily dose of dapagliflozin is 4.5 mg. In some embodiments, the total daily dose of dapagliflozin is 5.0 mg. In some embodiments, the total daily dose of dapagliflozin is 5.5 mg. In some embodiments, the total daily dose of dapagliflozin is 6.0 mg. In some embodiments, the total daily dose of dapagliflozin is 6.5 mg. In some embodiments, the total daily dose of dapagliflozin is 7.0 mg. In some embodiments, the total daily dose of dapagliflozin is 7.5 mg. In some embodiments, the total daily dose of dapagliflozin is 8.0 mg. In some embodiments, the total daily dose of dapagliflozin is 8.5 mg. In some embodiments, the total daily dose of dapagliflozin is 9.0 mg. In some embodiments, the total daily dose of dapagliflozin is 9.5 mg. In some embodiments, the total daily dose of dapagliflozin is 10.0 mg.

In some embodiments, zibotentan, or a pharmaceutically acceptable salt thereof, is administered once daily in a fixed-dose combination with dapagliflozin, or a pharmaceutically acceptable salt thereof.

In some embodiments, a fixed-dose combination of zibotentan and dapagliflozin is administered comprising a total daily dose of 0.2 mg to 5 mg of zibotentan or a pharmaceutically acceptable salt thereof and a total daily dose of 2.5 mg to 10.0 mg of dapagliflozin or a pharmaceutically acceptable salt thereof. In some embodiments, a fixed-dose combination of zibotentan and dapagliflozin is administered comprising a total daily dose of 0.25 mg of zibotentan or a pharmaceutically acceptable salt thereof and a total daily dose of 10.0 mg of dapagliflozin or a pharmaceutically acceptable salt thereof. In some embodiments, a fixed-dose combination of zibotentan and dapagliflozin is administered comprising a total daily dose of 0.5 mg of zibotentan or a pharmaceutically acceptable salt thereof and a total daily dose of 10.0 mg of dapagliflozin or a pharmaceutically acceptable salt thereof. In some embodiments, a fixed-dose combination of zibotentan and dapagliflozin is administered comprising a total daily dose of 0.75 mg of zibotentan or a pharmaceutically acceptable salt thereof and a total daily dose of 10.0 mg of dapagliflozin or a pharmaceutically acceptable salt thereof. In some embodiments, a fixed-dose combination of zibotentan and dapagliflozin is administered comprising a total daily dose of 1.0 mg of zibotentan or a pharmaceutically acceptable salt thereof and a total daily dose of 10.0 mg of dapagliflozin or a pharmaceutically acceptable salt thereof. In some embodiments, a fixed-dose combination of zibotentan and dapagliflozin is administered comprising a total daily dose of 1.5 mg of zibotentan or a pharmaceutically acceptable salt thereof and a total daily dose of 10.0 mg of dapagliflozin or a pharmaceutically acceptable salt thereof. In some embodiments, a fixed-dose combination of zibotentan and dapagliflozin is administered comprising a total daily dose of 5 mg of zibotentan or a pharmaceutically acceptable salt thereof and a total daily dose of 10.0 mg of dapagliflozin or a pharmaceutically acceptable salt thereof.

In some embodiments, administration of the fixed-dose combination of zibotentan, or a pharmaceutically acceptable salt thereof, and dapagliflozin, or a pharmaceutically acceptable salt thereof to a patient in need thereof results in partial remission or remission of liver fibrosis. In some embodiments, administration of the fixed-dose combination of zibotentan, or a pharmaceutically acceptable salt thereof, and dapagliflozin, or a pharmaceutically acceptable salt thereof to a patient in need thereof reduces the patient's liver fibrosis. In some embodiments, administration of the fixed-dose combination of zibotentan, or a pharmaceutically acceptable salt thereof, and dapagliflozin, or a pharmaceutically acceptable salt thereof to a patient in need thereof reduces the patient's liver fibrosis and reduces the patient's UACR.

In some embodiments, the improvement in disease, reduction of incidence, reduction of risk, and other beneficial effects described herein and provided for through administration of the fixed-dose combination of zibotentan and dapagliflozin as described in the preceding embodiments can represent improvements relative to the absence of therapy, improvements relative to placebo treatment, improvements relative to treatment with dapagliflozin alone, and/or improvements relative to treatment with other standard treatments for CKD and liver fibrosis and/or diseases, disorders, or conditions associated with CKD and liver fibrosis.

In some embodiments, the fixed-dose combination of zibotentan, or a pharmaceutically acceptable salt thereof, and dapagliflozin, or a pharmaceutically acceptable salt thereof, are administered concurrently. In some embodiments, zibotentan, or a pharmaceutically acceptable salt thereof, is administered prior to administration of dapagliflozin, or a pharmaceutically acceptable salt thereof. In some embodiments, zibotentan, or a pharmaceutically acceptable salt thereof, is administered after administration of dapagliflozin, or a pharmaceutically acceptable salt thereof.

Pharmaceutical compositions may be administered in any manner appropriate to the disease, disorder, and/or condition to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical composition can be formulated employing conventional solid or liquid vehicles, diluents, and pharmaceutical additives as appropriate for the mode of desired administration. The pharmaceutical compositions can be administered by a variety of routes including, for example, orally (e.g., in the form of tablets, capsules, granules, powders, and the like), parenterally (e.g., in the form of injectable preparations), intranasally, rectally, and transdermally (e.g., in the form of patches, for example).

The above dosage forms can also include a pharmaceutically acceptable carrier (i.e., a non-toxic, inert solid, semi-solid, or liquid filler, diluent, encapsulating material, or formulation auxiliary of any type), excipient, lubricant, buffer, antibacterial, bulking agent (such as mannitol), adjuvant, and the like.

Some examples of materials which can serve as pharmaceutically acceptable carriers are sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such a propylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions, as well as other non-toxic compatible lubricants, such as sodium lauryl sulfate and magnesium stearate; coloring agents; releasing agents; coating agents; sweetening; flavoring; perfuming agents; preservatives; and antioxidants.

Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters, such as ethyl oleate. Proper fluidity can be maintained, for example, by the use of a coating such as lecithin, by the maintenance of the required particle size in the case of dispersions, and by the use of surfactants.

Examples of adjuvants include preservative agents, wetting agents, emulsifying agents, dispersing agents, suspending agents, sweetening, flavoring, and perfuming agents. Prevention of the action of microorganisms can be ensured by various antibacterial and antifungal agents, for example, parabens, chlorobutanol, phenol, sorbic acid, and the like. It can also be desirable to include isotonic agents, for example, sugars, sodium chloride, and the like. Prolonged absorption of the injectable pharmaceutical form can be brought about by the use of agents delaying absorption, for example, aluminum monostearate and gelatin. Suspending agents include, for example, ethoxylated isostearyl alcohols, polyoxyethylene sorbitol, sorbitan esters, microcrystalline cellulose, aluminum metahydroxide, bentonite, agar-agar, tragacanth, and mixtures thereof.

The various pharmaceutical compositions employed in the methods of the disclosure can optionally include one or more fillers or excipients in an amount within the range of from about 0% to about 90% by weight and in some embodiments from about 1% to about 80% by weight. Examples of suitable fillers or excipients include, but are not limited to, lactose, sugar, corn starch, modified corn starch, mannitol, sorbitol, inorganic salts, such as calcium carbonate, and cellulose derivatives, such as wood cellulose and microcrystalline cellulose.

One or more binders can be present in addition to or in lieu of the fillers in an amount within the range of from about 0% to about 35%. In some embodiments, the binders are present in an amount of from about 0.5% to about 30% by weight of the composition. Examples of suitable binders include polyvinylpyrrolidone (molecular weight ranging from about 5000 to about 80,000 and in some embodiments about 40,000), lactose, starches, such as corn starch, modified corn starch, sugars, gum acacia, and the like, as well as a wax binder in finely powdered form (less than 500 microns), such as carnauba wax, paraffin, spermaceti, polyethylenes, and microcrystalline wax.

In some embodiments, the pharmaceutical composition is in the form of a tablet, wherein the tablet includes one or more tableting lubricants in an amount within the range of from about 0.2% to about 8% by weight of composition. In some embodiments, the tableting lubricant(s) is in an amount within the range of from about 0.5% to about 2% by weight of the composition. Examples of suitable tableting lubricants include, but are not limited to, magnesium stearate, stearic acid, palmitic acid, calcium stearate, talc, carnauba wax, and the like. Other ingredients can optionally be present, including, for example, preservatives, stabilizers, colorants, anti-adherents and silica flow conditioners, or glidants, such as Syloid brand silicon dioxide.

In some embodiments, the pharmaceutical composition is in the form of a tablet, wherein the tablet includes a coating layer which can comprise from about 0% to about 15% by weight of the tablet composition. The coating layer can comprise any conventional coating formulations that can include, for example, one or more film-formers or binders and/or one or more plasticizers. Examples of suitable film-formers or binders include, but are not limited to, hydrophilic polymers, such as hydroxypropylmethylcellulose, hydrophobic polymers, such as methacrylic acid esters, neutral polymers, ethyl cellulose, cellulose acetate, polyvinyl alcohol-maleic anhydride copolymers, β-pinene polymers, glyceryl esters of wood resins, and the like. Examples of suitable plasticizers include, but are not limited to, triethyl citrate, diethyl phthalate, propylene glycol, glycerin, butyl phthalate, castor oil, and the like. Both core tablets as well as coating formulations can contain aluminum lakes to provide color.

In some embodiments, the pharmaceutical composition is in the form of a tablet, wherein film-formers are applied to the tablet from a solvent system containing one or more solvents including water, alcohols such as methyl alcohol, ethyl alcohol, and isopropyl alcohol, ketones such as acetone and ethylmethyl ketone, or chlorinated hydrocarbons such as methylene chloride, dichloroethane, and 1, 1, 1-trichloroethane.

In some embodiments, the pharmaceutical composition is in the form of a tablet, wherein color is applied together with the film former, plasticizer, and solvent compositions.

In some embodiments, the pharmaceutical composition for use in the methods of the disclosure in the form of a tablet can be obtained by a process comprising the steps of:
a) mixing the inactive ingredients with the at least one compound of Formula (I);
b) formulating granules;
c) drying and/or screening the granules;
d) blending the granules; and
e) tableting the blend obtained in (d) into tablets.

In some embodiments, step a) of the process employs impact blending or milling and/or sizing equipment. In some embodiments, the granules in step b) of the process are formulated by dry granulation, wet granulation, or direct compression. In some embodiments, the granules are formulated by dry granulation. In some embodiments, the granules in step d) of the process are blended with a tableting aid or a lubricant and filler.

In some embodiments, the pharmaceutical composition in the form of a capsule can be obtained by a process comprising the steps of:
a) mixing the inactive ingredients with the at least one compound of Formula (I) using a combination of blending and milling processes;
b) formulating granules;
c) drying and/or screening the granules; and
d) loading the granules into capsules.

In some embodiments, step a) of the process employs impact milling or blending and/or sizing equipment. In some embodiments, the granules in step b) of the process are formulated by dry granulation, wet granulation, or direct compression. In some embodiments, the granules are formulated by dry granulation.

In some embodiments, the pharmaceutical composition may also contain additional ingredients, such as adjuvants, preservatives, wetting agents, emulsifying agents, and dispersing agents. Prevention of the action of microorganisms can be facilitated by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form can be brought about by the inclusion of agents which delay absorption such as aluminum monostearate and gelatin.

In some embodiments, it is desirable to slow the absorption of the drug from subcutaneous or intramuscular injection. In some embodiments, this is accomplished by the use of a liquid suspension of crystalline or amorphous material with poor water solubility. The rate of absorption of the drug then depends upon its rate of dissolution which, in turn, may depend upon crystal size and crystalline form. In some embodiments, delayed absorption is accomplished by dissolving or suspending the drug in an oil vehicle.

In some embodiments, the pharmaceutical composition is in an injectable depot form. In some embodiments, the injectable depot form comprises microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers suitable for use herein include poly(orthoesters) and poly(anhydrides). In some embodiments, depot injectable formulations are prepared by entrapping the drug in liposomes or microemulsions which are compatible with body tissues.

In some embodiments, the pharmaceutical composition is an injectable formulation, wherein the injectable formulation may be sterilized, for example, by filtration through a bacterial-retaining filter or by incorporating sterilizing agents in the form of sterile solid compositions which can be dissolved or dispersed in sterile water or other sterile injectable medium just prior to use.

In some embodiments, the pharmaceutical composition is a solid dosage form suitable for oral administration. Solid dosage forms for oral administration include capsules, tablets, pills, powders, and granules. In some embodiments, the at least one compound chosen from compounds disclosed herein and prodrugs thereof is mixed with at least one inert, pharmaceutically acceptable excipient or carrier, such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay, and/or i) lubricants such as talc, calcium stearate, magnesium stearate, solid polyethylene glycols, sodium lauryl sulfate, and mixtures thereof. In some embodiments, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

In some embodiments, tablets, dragees, capsules, pills, and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

In some embodiments the at least one compound chosen from the compounds disclosed herein and/or prodrugs thereof may be in micro-encapsulated form, if appropriate, with one or more of the above-mentioned excipients.

In some embodiments the pharmaceutical composition may be in liquid dosage form suitable for oral administration including pharmaceutically acceptable emulsions, solutions, suspensions, syrups, and elixirs. In some embodiments, the liquid dosage form may contain inert diluents commonly used in the art such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols, and fatty acid esters of sorbitan, and mixtures thereof.

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### EXAMPLES

### Example 1

Clinical trials of the fixed-dose combination of dapagliflozin and zibotentan are ongoing, including a randomized, double-blind, placebo-controlled, parallel group-dose ranging study to assess the efficacy of fixed-dose combination of dapagliflozin and zibotentan compared to dapagliflozin monotherapy and placebo in patients with chronic kidney disease (the “ZENITH-CKD” trial). The ZENITH-CKD trial is described in, for example, U.S. Pat. No. 11,730,735, issued Aug. 22, 2023, the contents of which are incorporated by reference herein in its entirety.

The efficacy of a fixed-dose combination of zibotentan and dapagliflozin was assessed in a subset of patients from the ZENITH-CKD trial that had CKD and were suspected of having advanced liver fibrosis (NFS>0.675). Two different combinations of zibotentan and dapagliflozin were evaluated where zibotentan was administered at a dose of 0.25 mg or 1.5 mg, and dapagliflozin was administered at a dose of 10 mg. The combinations of zibotentan and dapagliflozin (0.25 mg zibotentan and 10 mg dapagliflozin; 1.5 mg zibotentan and 10 mg dapagliflozin) were administered once daily over a 12-week period. NFS was also calculated at a safety follow-up visit at week 14 (post-washout), following completion of the twelve-week dosing period.

The endpoints in this study were (i) the change in UACR at week 12, relative to baseline; (ii) the change in NFS at week 12, relative to baseline; and (iii) the change in NFS at week 14 (post-washout), relative to baseline. The individual components of the NFS were also measured at week 12, relative to baseline. Biomarker analyses were also conducted to assess the effect on insulin resistance, inflammatory biomarkers, and lipid biomarkers.

The NFS is a composite score that estimates the amount of scarring in the liver based on a number of parameters and laboratory tests (Angulo P et al., The NAFLD fibrosis score: a noninvasive system that identifies liver fibrosis in patients with NAFLD. Hepatology. 2007; 45:846-854). The NFS calculation accounts for the subject's age, body mass index, hyperglycemia, platelet count, albumin level, and aspartate aminotransferase level/alanine aminotransferase level (AST/ALT) ratio. A NFS of greater than 0.675 is indicative of a high risk for advanced fibrosis.

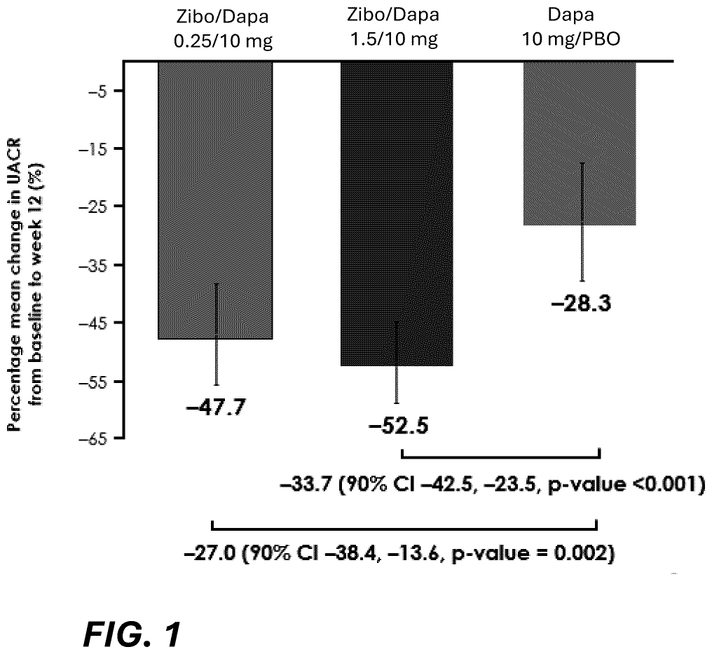

A total of 39 patients having an NFS>0.675 received the combination therapy of zibotentan and dapagliflozin (either dose) and a total of 27 patients having an NFS>0.675 were administered dapagliflozin alone. Median baseline NFS calculations, median NFS reduction at week 12 and week 14 assessments, and measurements for the individual components of the NFS are detailed in Table 1. The mean change (percent) in UACR from baseline to week 12 is shown in FIG. 1.

TABLE 1
Median (Q1, Q3) NFS change for study participants.

| | | | |
|---|---|---|---|
| Zibotentan | Zibotentan | Zibotentan + | Placebo + |
| (0.25 mg) + | (1.5 mg) + | Dapagliflozin | Dapagliflozin |
| Dapagliflozin | Dapagliflozin | (both groups) | (10 mg) |
| (10 mg) (n = 10) | (10 mg) (n = 29) | (n = 39) | (n = 27) |

Overall NFS
Baseline

| | | | | |
|---|---|---|---|---|
| Median (Q1, | 1.03 (0.93, | 1.37 (0.83, | 1.12 (0.83, | 1.21 (0.99, |
| Q3) | 1.23) | 1.77) | 1.58) | 1.62) |
| n | 6 | 24 | 30 | 20 |
| Median (Q1, | −0.56 (−0.69, | −0.09 (−0.57, | −0.13 (−0.50, | −0.26 (−0.54, |
| Q3) | −0.40) | −0.08) | 0.08) | 0.30) |

Change from Baseline to Week 14

| | | | | |
|---|---|---|---|---|
| N | 5 | 21 | 26 | 22 |
| Median (Q1, | −0.78 (−0.95, | −0.26 (−0.57, | −0.33 (−0.76, | −0.12 (−0.73, |
| Q3) | −0.70) | −0.08) | −0.08) | 0.03) |

Change from Baseline to Week 12 in NFS Components (Median, (Q1, Q3))

| | | | | |
|---|---|---|---|---|
| Albumin (g/dL) | 0.10 (−0.20, | 0.00 (−0.10, | 0.00 (−0.10, | 0.05 (−0.20, |
| | 0.30) | 0.25) | 0.30) | 0.10) |
| Platelets | 21.00 (−5.00, | −2.00 (−21.50, | −1.00 (−15.00, | 2.50 (−24.50, |
| (10⁹/L) | 46.00) | 12.50) | 30.00) | 25.00) |
| AST-ALT Ratio | −0.02 (−0.17, | 0.05 (−0.11, | 0.04 (−0.13, | −0.03 (−0.29, |
| | 0.04) | 0.22) | 0.20) | 0.18) |
| AST (U/L) | 4.00 (−1.00, | −1.00 (−4.50, | −1.00 (−4.00, | −2.00 (−4.50, |
| | 7.00) | 1.00) | 1.00) | 1.00) |
| ALT (U/L) | 1.50 (0.00, | −3.00 (−5.50, | −2.50 (−5.00, | −0.50 (−4.00, |
| | 5.00) | 2.00) | 2.00) | 3.00) |
| Body Mass | −1.00 (−1.30, | −0.40 (−0.75, | −0.40 (−1.00, | −0.10 (−0.40, |
| Index | 0.00) | 0.00) | 0.00) | 0.45) |

In this smaller cohort of patients, a reduction in NFS was observed in patients having CKD with high risk for advanced liver fibrosis over twelve weeks, following the administration of a combination of zibotentan and dapagliflozin. Notably, patients receiving the lower dose combination of zibotentan and dapagliflozin (0.25 mg and 10 mg, respectively), exhibited the greatest reduction in NFS at both week 12 and week 14 assessments, with a median reduction in NFS of 0.56 and 0.78, respectively. Patients receiving the higher dose combination of zibotentan and dapagliflozin (1.5 mg and 10 mg, respectively) still exhibited a reduction in NFS at week 12 and week 14 assessments (0.09 and 0.26, respectively), albeit at a lower level relative to the lower dose combination treatment. Administration of dapagliflozin alone also reduced median NFS at both week 12 (0.26) and week 14 (0.21) assessments. Evaluation of the individual NFS components indicated that the lower dose combination of zibotentan and dapagliflozin (0.25 mg/10 mg, respectively) increased platelet count and serum albumin levels. The higher dose combination of zibotentan and dapagliflozin (1.5 mg/10 mg, respectively) reduced AST and ALT levels.

Without being bound by any particular theory, the slightly decreased reduction in NFS recorded when administering the higher-dose combination of zibotentan and dapagliflozin may be due to fluid retention associated with the higher dose of zibotentan, which would have a dilutional effect on the platelet count and serum albumin parameters of the NFS calculation.

Both the lower dose combination of zibotentan and dapagliflozin (0.25 mg and 10 mg, respectively) and the higher dose combination of zibotentan and dapagliflozin (1.5 mg and 10 mg, respectively) demonstrated a greater effect in the mean change (percentage) in UACR relative to patients receiving dapagliflozin alone (10 mg) (FIG. 1). Patients receiving the lower dose combination of zibotentan and dapagliflozin (0.25 mg and 10 mg, respectively) demonstrated a mean change in UACR from baseline of −47.7%, and patients receiving the higher dose combination of zibotentan and dapagliflozin (1.5 mg and 10 mg, respectively) demonstrated a mean change in UACR from baseline of −52.5%, relative to a −28.3% change from baseline in patients receiving dapagliflozin alone (10 mg). The mean percent change in UACR from baseline for patients receiving both the lower dose and higher dose of the combination of zibotentan and dapagliflozin was statistically significant (p=0.002 and p<0.0001, respectively) compared to the mean change in UACR in patients receiving dapagliflozin alone.

Because a NFS of >0.675 corresponds to a high risk for advanced fibrosis, the reductions in NFS suggest that, in addition to the impact of the combination of zibotentan and dapagliflozin on UACR in this population, the combination of zibotentan and dapagliflozin has additional benefits beyond renoprotection and can be effective in treating and/or protecting against liver fibrosis.

A biomarker analysis was conducted in subjects from this study (N=237; 138 without diabetes, 99 with type II diabetes) to evaluate the impact of the combination of zibotentan and dapagliflozin on insulin resistance and markers of inflammation. Differences in changes from baseline were assessed for insulin resistance using the homeostatic model assessment (HOMA2-IR; see Levy J C, Matthews D R, Hermans M P. Correct homeostasis model assessment (HOMA) evaluation uses the computer program. Diabetes Care. 1998; 21 (12): 2191-2192), and changes from baseline in inflammatory biomarkers MCP-1, IL-6, and hsCRP, were measured in plasma. Participants using insulin were excluded from this analysis.

Baseline levels for all measures were balanced across the three treatment groups evaluated, with patients having median baseline levels of 3, 0.9 pg/mL, 146 μg/ml, and 1.3 mg/L for HOMA2-IR, MCP-1, IL-6, and hsCRP, respectively. Table 2 shows the changes from baseline in analyte levels for the three treatment groups. Notably treatment with both doses of the combination of zibotentan and dapagliflozin (0.25/10 mg and 1.5/10 mg) resulted in statistically significant reductions in HOMA2-IR relative to baseline when compared to the dapagliflozin treatment group. The reduction in HOMA2-IR levels relative to baseline was consistent regardless of diabetic status. The combination of zibotentan and dapagliflozin also reduced levels of inflammatory biomarkers relative to baseline, with statistically significant reductions in levels of MCP-1 and IL-6 after 12-weeks relative to baseline when compared to patients receiving dapagliflozin. The high dose combination of zibotentan and dapagliflozin (1.5/10 mg) also demonstrated a statistically significant reduction in IL-6 relative to baseline when compared against patients receiving dapagliflozin.

TABLE 2
Change from baseline in HOMA2-IR and inflammatory biomarkers.

| | | Change from | | Difference vs | |
|---|---|---|---|---|---|
| | | Baseline | | Dapagliflozin | |
| Measure | Treatment Group | (% [90% CI]) | | (% [90% CI]) | |
| HOMA2-IR | Dapa 10 mg | −1.3 | (−11.2, 9.7) | — | |
| | Zibo/Dapa 0.25/10 mg | −20.3 | (−29.4, −10.1) | −19.3 | (−28.9, −8.4) |
| | | | | p = 0.05 | |
| | Zibo/Dapa 1.5/10 mg | −15.9 | (−24.4, −6.5) | −14.8 | (−23.2, −5.6) |
| | | | | p = 0.011 | |
| MCP-1 | Dapa 10 mg | −0.8 | (−11.2, 10.7) | — | |
| | Zibo/Dapa 0.25/10 mg | −11.7 | (−22.5, 0.6) | −10.9 | (−22.1, 1.8) |
| | | | | p = 0.152 | |
| | Zibo/Dapa 1.5/10 mg | −18.1 | (−26.6, −8.6) | −17.4 | (−26.0, −7.9) |
| | | | | p = 0.004 | |
| IL-6 | Dapa 10 mg | 7.8 | (1.6, 14.5) | — | |
| | Zibo/Dapa 0.25/10 mg | −1.0 | (−7.5, 6.0) | −8.2 | (−14.4, −1.5) |
| | | | | p = 0.046 | |
| | Zibo/Dapa 1.5/10 mg | −7.7 | (−13.0, −2.2) | −14.5 | (−19.3, −9.4) |
| | | | | p < 0.001 | |
| hsCRP | Dapa 10 mg | −14.5 | (−31.4, 6.5) | — | |
| | Zibo/Dapa 0.25/10 mg | −6.6 | (−27.0, 19.5) | 9.2 | (−16.2, 42.4) |
| | | | | p = 0.584 | |
| | Zibo/Dapa 1.5/10 mg | −22.9 | (−38.3, −3.6) | −9.8 | (−27.8, 12.8) |

p = 0.447
dapa = dapagliflozin,
zibo = zibotentan

The combination of zibotentan and dapagliflozin at both doses tested (0.5/10 mg and 1.5/10 mg) resulted in a reduction in insulin resistance (shown by HOMA2-IR) and also resulted in reduced levels of inflammation in patients, irrespective of diabetic status. Without being bound by any theory, the reduction in insulin resistance and inflammatory biomarkers associated with administration of the combination of zibotentan and dapagliflozin may contribute to long-term kidney and cardiovascular protection, as well as the treatment of eGFR decline and other CKD-related clinical outcomes.

A lipid biomarker analysis was also conducted in subjects from this study (N=237; 138 without diabetes, 99 with type II diabetes) to evaluate the impact of the combination of zibotentan and dapagliflozin on lipid biomarkers. Because a dose-dependent effect was not observed between the two different treatment groups receiving the combination of zibotentan and dapagliflozin (0.25/10 mg and 1.5/10 mg), data for subjects receiving the combination of zibotentan and dapagliflozin was combined (n=220) and compared against subjects receiving dapagliflozin alone (10 mg; n=148). Changes from baseline after twelve weeks of treatment in proprotein convertase subtilisin/kexin type 9 (PCSK9), lipoprotein (a) (Lp(a)), low-density lipoprotein cholesterol (LDL-C), apolipoprotein B (ApoB), and the LDL-C/ApoB ratio were evaluated. At baseline, lipid biomarker levels were similar across the groups (Table 3). When compared with dapagliflozin, zibotentan/dapagliflozin combination therapy significantly lowered levels of PCSK9 (−8.74%; 90% Cl: −12.40, −4.92), Lp(a) (−8.20%; 90% Cl: −13.92, −2.10), and ApoB (−7.36%; 90% Cl: −10.63, −3.97). A reduction in LDL-C was observed but did not reach statistical significance, and a numerical increase in LDL-C/ApoB ratio was likewise observed (Table 3).

TABLE 3
Change from baseline in lipid biomarkers.

| | | | Change from | |
|---|---|---|---|---|
| | Treatment | Baseline | baseline | Difference vs Dapa |
| Biomarker | Group | level | (% [90% CI]) | (% [90% CI]) |
| PCSK9 | Dapa | 267.9 | −5.10% (−9.54, | — |
| (ng/mL) | | (142.2) | −0.44) | |
| | Zibo + | 283.4 | −13.40% (−17.17, | −8.74% (−12.40, |
| | Dapa | (251.1) | −9.45) | −4.92) |
| Lp(a) | Dapa | 164.5 (75.5- | −0.83% (−8.45, | — |
| (mg/L) | | 301.0) | 7.42) | |
| | Zibo + | 179.0 (75.0- | −8.96% (−15.40, | −8.20% (−13.92, |
| | Dapa | 391.0) | −2.03) | −2.10) |
| LDL-C | Dapa | 2.2 | 0.44% (−5.47, | — |
| (mmol/L) | | (1.0) | 6.71) | |
| | Zibo + | 2.2 | −3.84% (−9.04, | −4.26% (−9.39, |
| | Dapa | (1.0) | 1.67) | 1.17) |
| ApoB | Dapa | 0.9 | 4.30% (0.24, | — |
| (g/L) | | (0.3) | 8.54) | |
| | Zibo + | 0.9 | −3.37% (−6.90, | −7.36% (−10.63, |
| | Dapa | (0.3) | 0.29) | −3.97) |
| LDL- | Dapa | 2.5 | −2.02% (−6.66, | |
| C/ApoB | | (0.6) | 2.85) | |
| ratio | Zibo + | 2.5 | 1.73% (−2.67, | 3.83% (−0.66, |
| | Dapa | (0.7) | 6.34) | 8.52) |

Baseline levels: Data are mean (SD) or median (IQR), as appropriate;
dapa = dapagliflozin,
zibo = zibotentan

Without being bound by any theory, the combination of zibotentan and dapagliflozin at both doses tested (0.5/10 mg and 1.5/10 mg) improved key lipid parameters, notably reducing PCSK9, Lp(a) and ApoB levels, compared to dapagliflozin alone, suggesting potential cardiovascular benefits beyond albuminuria reduction.

1. A method of treating liver fibrosis in a human patient who has chronic kidney disease (CKD), the method comprising administering to the patient a fixed-dose combination of zibotentan and dapagliflozin in an amount effective to treat the patient's liver fibrosis.

2. The method of claim 1, wherein the patient has CKD and fibrosis.

3-22. (canceled)

23. The method according to claim 1, wherein the patient is naïve to a sodium-glucose cotransporter-2 (SGLT2) inhibitor.

24. The method according to claim 1, wherein the patient has an estimated glomerular filtration rate (eGFR) of 20-90 mL/min/1.73 m².

25. The method according to claim 1, comprising administering the fixed-dose combination of zibotentan and dapagliflozin to the patient once per day.

26. The method according to claim 1, comprising administering zibotentan at a dose of 0.25 mg to 5 mg.

27-33. (canceled)

34. The method according to claim 1, comprising administering dapagliflozin at a dose of 2.5 mg to 10 mg.

35-40. (canceled)

41. The method according to claim 1, comprising administering zibotentan at a dose of 0.25 mg and dapagliflozin at a dose of 10 mg when the patient has an eGFR that is <45 mL/min/1.73 m², and administering zibotentan at a dose of 1.5 mg and dapagliflozin at a dose of 10 mg when the patient has an eGFR that is ≥45 mL/min/1.73 m².

42. The method according to claim 41, wherein:
(i) the fixed dose combination of zibotentan and dapagliflozin is adjusted from 1.5 mg of zibotentan and 10 mg of dapagliflozin to 0.25 mg of zibotentan and 10 mg of dapagliflozin when the patient's eGFR changes to <45 mL/min/1.73 m²; or
(ii) the fixed dose combination of zibotentan and dapagliflozin is adjusted from 0.25 mg of zibotentan and 10 mg of dapagliflozin to 1.5 mg of zibotentan and 10 mg of dapagliflozin when the patient's eGFR changes to >45 mL/min/1.73 m².

43. (canceled)

44. The method according to claim 1, wherein administration of the fixed-dose combination of zibotentan and dapagliflozin reduces the patient's nonalcoholic fatty liver disease (NAFLD) fibrosis score (NFS) to below 0.675.

45. The method according to claim 1, wherein the patient achieves partial remission or remission.

46. The method according to claim 1, wherein administration of the fixed-dose combination of zibotentan and dapagliflozin does not increase the patient's NFS relative to a dosing regimen in which the patient receives dapagliflozin alone.

47. The method according to claim 1, wherein administration of the fixed-dose combination of zibotentan and dapagliflozin reduces the risk of increased NFS in the patient relative to a dosing regimen in which the patient receives dapagliflozin alone.

48. The method according to claim 1, wherein administration of the fixed-dose combination of zibotentan and dapagliflozin reduces the patient's liver fibrosis relative to a dosing regimen in which the patient receives dapagliflozin alone.

49. The method according to claim 1, wherein administration of the fixed-dose combination of zibotentan and dapagliflozin reduces the patient's liver fibrosis and reduces the patient's UACR relative to a dosing regimen in which the patient receives dapagliflozin alone.

50. The method according to claim 1, wherein administration of the fixed-dose combination of zibotentan and dapagliflozin increases (i) the patient's platelet count, serum albumin, or both platelet count and serum albumin; and/or (ii) the patient's aspartate aminotransferase (AST) level, alanine aminotransferase (ALT) level, or both AST and ALT levels.

51. (canceled)

52. The method according to claim 1, wherein the method reduces levels of one or more of MCP-1, IL-6, and high-sensitivity C-reactive protein (hsCRP).

53. (canceled)

54. The method according to claim 1, wherein the method results in a reduction in insulin resistance as shown by assessment of Homeostatic Model Assessment version 2-Insulin Resistance (HOMA2-IR).

55. The method according to claim 1, wherein the method reduces levels of one or more of proprotein convertase subtilisin/kexin type 9 (PCSK9), lipoprotein (a) (Lp(a)), low-density lipoprotein cholesterol (LDL-C), and apolipoprotein B (ApoB).

56. The method according to claim 55, wherein the low-density lipoprotein cholesterol (LDL-C)/Apolipoprotein B (ApoB) ratio is increased.
